(19) United States
(12) Patent Application Publication
(10) Pub. No.: US 2021/0121514 A1
(43) Pub. Date: Apr. 29, 2021

(54) METHODS AND COMPOSITIONS FOR TREATING AND PREVENTING MUSCLE SORENESS AND FOOT & JOINT PAIN

(71) Applicant: Spraingo, LLC, Brandon, FL (US)

(72) Inventor: Wayne Caleb Williams, Brandon, FL (US)

(21) Appl. No.: 16/995,310

(22) Filed: Aug. 17, 2020

Related U.S. Application Data
(60) Provisional application No. 62/924,857, filed on Oct. 23, 2019.

(51) Int. Cl. A61K 36/15; A61K 9/00; A61K 9/06; A61K 31/05; A61K 31/19; A61K 36/28; A61K 33/06

(52) U.S. Cl. CPC A61K 36/15; A61K 9/0014; A61K 9/06; A61K 33/06; A61K 31/19; A61K 36/28; A61K 31/05

(57) ABSTRACT

The present disclosure provides methods for treating and preventing foot pain, muscle soreness, inflammation or an inflammatory-related disorder, and itch with the administration of compositions comprising pine resin. Also provided are methods of repelling an insect by applying to a subject composition comprising pine resin.

### CROSS-REFERENCE TO RELATED APPLICATIONS

This application claims the benefit of U.S. Provisional Application No. 62/924,857, filed Oct. 23, 2019, the content of which is incorporated herein by reference.

### FIELD

The present disclosure provides methods for treating and preventing muscle soreness foot pain, inflammation or inflammatory-related disorders, or itch.

### BACKGROUND

Millions of individuals every year purchase over the counter and prescription medicines for relief of pain, inflammation and itch. For serious and chronic conditions or recovery from medical procedures, health professionals often prescribe prescription painkillers, steroids, or anti-inflammatories. For routine, acute or common injuries, inflammation, or itches, however, it is common for individuals to take oral analgesics or apply a topical analgesic (spray, lotions, ointments, or pain patches) to the affected areas.

Insects, particularly ticks and mosquitoes, are a perennial problem and are often regarded as pests to both animals and humans. Insects can carry many different diseases and their bites can cause itching and/or discomfort in both animals and humans and can also result in an infection of the bitten area. In addition, many humans and animals have a high sensitivity to the bites or infestations of various insects and may suffer serious allergic reactions and even death due to insects. Moreover, with regard to livestock and pets, insects can create havoc and insect-related problems which may have serious economic effects. Consequently, compositions which repel or kill insects are commonly used to keep insects away from animals and humans. However, the harsh chemicals typically contained in currently available insect repellants have been known to cause significant skin irritation when they are applied to a living surface, such as the skin of animals or humans.

### SUMMARY

Disclosed herein are methods for treating and preventing a disease or disorder selected from the group consisting of: muscle soreness, foot pain, inflammation or an inflammatory-related condition, and itch. The methods comprise administering to a subject a composition comprising 1% to 25% by weight pine resin. The composition may be administered before or after the onset of the muscle soreness, foot pain, inflammation or an inflammatory-related condition, or itch.

Also disclosed herein are methods of repelling an insect. The methods comprise applying to a subject, in an amount effective to repel the insect, a composition comprising 1% to 25% by weight pine resin. In some embodiments, the composition may be applied to the skin of the subject. In some embodiments, the composition may be applied to an article, wherein the article is worn or inhabited by the subject.

The compositions may further comprise: at least one Arnica extract; at least one extract derived from the group consisting of Harpagopthum, Bryophyllus pinnatum, Calendula officinalis, Boswellia serrata, Cinnamomum camphora, Mentha piperita, Ruta graveolens, magnesium sulfate, Capsicum oleoresin, Symphytum officinale, magnesium sulfate, Capsicum oleoresin, and cannabidiol (CBD) and combinations thereof. In some embodiments, the composition is an ointment, cream, gel, lotion, spray, foam, paste, suspension or dispersion for topical administration.

Other aspects and embodiments of the disclosure will be apparent in light of the following detailed description and accompanying figures.

### DETAILED DESCRIPTION

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, “Arnica” is a perennial herb in the sunflower family which includes species such as A. chamissonis and A. montana.

As used herein, “abietic acid” is an abietane diterpene group of organic compounds derived from four isoprene units and may be referred to as abietinic acid, sylvic acid, abieta-7,13-dien-18-oic acid, or 1R,4aR,4bR,10aR)-7-isopropyl-1,4a-dimethyl-1,2,3,4,4a,4b,5,6,10,10a-decahydrophenanthrene-1-carboxylic acid. It is the most abundant of several closely related organic acids that constitute pine resin or rosin. The structure of abietic acid is:

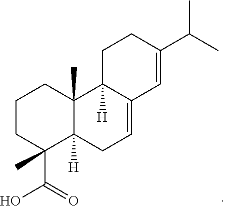

As used herein, “Boswellia serrata” refers to a plant that produces Indian frankincense.

As used herein, “Bryophyllum pinnatum” refers to a succulent plant native to Madagascar also known as kalanchoe pinnata, wonder of the world, the air plant, cathedral bells, life plant, miracle leaf, and Goethe plant.

As used herein, “Capsicum oleoresin” refers to an oily organic resin derived from the fruit of plants in the Capsicum genus, such as chili peppers. Capsicum oleoresin is formed after the extraction process of capsaicin using organic solvents such as ethanol. The intensity of biological actions and toxicological effects of Capsicum oleoresin are a direct function of the amount of capsaicinoids, or capsaicin, present. Capsaicinoids comprise of a group of fat-soluble pungent chemical phenols and include capsaicin, dihydrocapsaicin, nordihydrocapsaicin, homocapsaicin, and homodihydrocapsaicin.

“Calendula officinalis” is a short-lived aromatic herbaceous perennial also known as pot marigold, ruddles, common marigold or Scotch marigold.

“Cinnamomum camphora” or “camphora” is a species of evergreen tree that is commonly known as camphor tree, camphorwood or camphor laurel.

“Harpagophytum” also called grapple plant, wood spider and most commonly devil's claw, is a genus of plants in the sesame family.

“Mentha piperita” or peppermint is a hybrid mint herb cross between watermint and spearmint.

As used herein, the term “preventing” refers to partially or completely delaying onset of a disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

“Ruta graveolens” is an ornamental plant and herb commonly known as rue, common rue or herb-of-grace.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

“Symphytum officinale” is one of 34 species of Symphytum, and is commonly referred to as common comfrey, true comfrey, Quaker comfrey, cultivated comfrey, boneset, knitbone, consound, and slippery-root.

Magnesium sulfate (magnesia suphurica) is naturally occurring compound comprising magnesium and sulfate in the formula MgSO₄ where it can be unhydrated or hydrated with up to seven water molecules. Most commonly, it is in the form of the heptahydrate magnesium sulfate mineral MgSO₄.7H₂O, commonly called Epsom salt.

As used herein, “treat,” “treating” and the like means a slowing, stopping or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Methods for Treating Muscle Soreness

The present disclosure provides methods for treating or preventing muscle soreness comprising administering to a subject a composition comprising about 1% to about 25% by weight pine resin.

a) Muscle Soreness

Muscle soreness may include any cause of soreness, including for example, tension, stress, overuse, minor injuries, or muscle cramps.

The muscle soreness may be a result of exercise-induced muscle damage. In some embodiments, the muscle soreness is delayed onset muscle soreness (DOMS). DOMS is the pain and stiffness felt in muscles several hours to days after unaccustomed or strenuous exercise. The soreness is usually felt most strongly about 24 to about 72 hours after the exercise and is thought to be caused by eccentric (lengthening) exercise, which causes small-scale damage (microtrauma) to the muscle fibers. The soreness in DOMs may also be referred to as muscular mechanical hyperalgesia.

In some embodiments, the muscle soreness is acute exercise-induced muscle soreness, which can appear during or immediately after strenuous exercise. The soreness or pain generally appears within a minute of contracting the muscle and disappears within two or three minutes or up to several hours after relaxing it.

The muscle soreness may be a result of muscle cramps. Muscle cramps are sudden, intense, electrically active contractions elicited by motor neuron hyperexcitability. Muscle cramps range in intensity from a slight tic to agonizing pain. A cramping muscle may feel hard to the touch and/or appear visibly distorted or twitch beneath the skin. A cramp can last from a few seconds to about 15 minutes or longer. Some people are pre-disposed to muscle cramps and get them regularly with or without any physical exertion. Severe muscle cramps can result in muscle soreness even after the cramping has stopped.

muscle soreness may be a result of a muscle sprain or strain. Muscle strains occur when an excessive amount of force or pressure is directed onto muscles that cause damage or tearing to the muscle fibers and/or surrounding tendons. Common muscle strains, torn muscles and pulled muscles are: Achilles tendon tear, pulled backs, lower back muscle strain, tearing the rotator cuff, abs (abdominal) muscle strains, calf muscle strain, hamstring muscle strain, quads (quadriceps) muscle strain, leg muscle strain, knee (or plantaris) muscle strain, chest muscle strain, groin pull or muscle strain, bicep muscle strain, and arm muscle strain. Sprains are caused by trauma such as a fall or blow to the body that knocks a joint out of position and, in the worst case, ruptures the supporting ligaments.

In some embodiments, the composition is administered before the onset of muscle soreness to prevent or delay the onset of muscle soreness. For example, the composition may be administered before an anticipated strenuous or long duration period of exercise. For example, in some embodiments, the composition may be administered at least 1 minute before exercise, at least 2 minutes before exercise, at least 3 minutes before exercise, at least 4 minutes before exercise, at least 5 minutes before exercise, at least 10 minutes before exercise, at least 15 minutes before exercise, at least 20 minutes before exercise, at least 30 minutes before exercise, at least 45 minutes before exercise, at least 1 hour before exercise, at least 2 hours before exercise, at least 3 hours before exercise, at least 4 hours before exercise, at least 5 hours before exercise, at least 6 hours before exercise, at least 7 hours before exercise, at least 8 hours before exercise, at least 9 hours before exercise, at least 10 hours before exercise, at least 11 hours before exercise, or at least 12 hours before exercise.

In some embodiments, the composition is administered after the onset of muscle soreness to relieve, improve or remove the muscle soreness. For example, in some embodiments, the composition is administered at least 1 minute after the onset of soreness, at least 2 minutes after the onset of soreness, at least 3 minutes after the onset of soreness, at least 4 minutes after the onset of soreness, at least 5 minutes after the onset of soreness, at least 10 minutes after the onset of soreness, at least 15 minutes after the onset of soreness, at least 20 minutes after the onset of soreness, at least 30 minutes after the onset of soreness, at least 45 minutes after the onset of soreness, at least 1 hour after the onset of soreness, at least 2 hours after the onset of soreness, at least 3 hours after the onset of soreness, at least 4 hours after the onset of soreness, at least 5 hours after the onset of soreness, at least 6 hours after the onset of soreness, at least 7 hours after the onset of soreness, at least 8 hours after the onset of soreness, at least 9 hours after the onset of soreness, at least 10 hours after the onset of soreness, at least 11 hours after the onset of soreness, or at least 12 hours after the onset of soreness. In some embodiments, the composition is administered before and after the onset of muscle soreness.

b) Compositions

The composition may include about 1 to about 25% by weight pine resin. The term “pine rosin,” “rosin,” and “gum rosin” may be synonymous with pine resin.

In some embodiments, the composition includes at least about 1% by weight pine resin, at least about 2% by weight pine resin, at least about 5% by weight pine resin, at least about 10% by weight pine resin, at least about 15% by weight pine resin, at least about 20% by weight pine resin, or at least about 25% by weight pine resin. In some embodiments, the composition includes less than about 25% by weight pine resin, less than about 20% by weight pine resin, less than about 15% by weight pine resin, less than about 10% by weight pine resin, less than about 5% by weight pine resin, or less than about 2% by weight pine resin. The composition may comprise about 1 to about 25% by weight pine resin, about 1 to about 20% by weight pine resin, about 1 to about 15% by weight pine resin, about 1 to about 10% by weight pine resin, about 1 to about 5% by weight pine resin, about 2 to about 25% by weight pine resin, about 2 to about 20% by weight pine resin, about 2 to about 15% by weight pine resin, about 2 to about 10% by weight pine resin, about 5 to about 25% by weight pine resin, about 5- to about 20% by weight pine resin, about 5 to about 15% by weight pine resin, about 5 to about 10% by weight pine resin, about 10 to about 25% by weight pine resin, about 10 to about 20% by weight pine resin, about 10 to about 15% by weight pine resin, about 15 to about 25% by weight pine resin, or about 15 to about 20% by weight pine resin. In certain embodiments, the composition comprises between about 5% and about 15% by weight pine resin.

Pine resin is a mixture of compounds, mainly composed of resin acids, lignans, terpenes and volatile fatty acid compounds. In some embodiments, the pine resin comprises at least about 30% abietic acid. The pine resin may comprise at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

In some embodiments, the pine resin comprises at least about 20% abietic acid. The pine resin may comprise at least about 21% abietic acid, at least about 22% abietic acid, at least about 23% abietic acid, at least about 24% abietic acid, at least about 25% abietic acid, at least about 26% abietic acid, at least about 27% abietic acid, at least about 28% abietic acid, at least about 29% abietic acid, at least about 30% abietic acid, at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 20 to about 60% abietic acid, about 20 to about 55% abietic acid, about 20 to about 50% abietic acid, about 20 to about 45% abietic acid, about 20 to about 40% abietic acid, about 20 to about 35% abietic acid, about 20 to about 30% abietic acid, about 20 to about 25% abietic acid, about 25 to about 60% abietic acid, about 25 to about 55% abietic acid, about 25 to about 50% abietic acid, about 25 to about 45% abietic acid, about 25 to about 40% abietic acid, about 25 to about 30% abietic acid, about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

Different species of pine may give rise to different mixtures of compounds in pine resin. In addition, the different species of pine grown in environmental conditions that impart different properties based on the particular growing region. The pine resin used in the composition may be derived from pine species from Central America, Caribbean islands (e.g., Cuba, the Bahamas, Turks and Caicos, Trinidad and Tobago, etc.) and Southeastern United States. As such, the pine resins used in the compositions of the present invention likely contain different components than those found in a pine or juniper plants found in Asia. In some embodiments, the pine resin is derived from Pinus caribaea (Caribbean pine), Pinus elliottii, or a combination thereof.

In some embodiments, the composition comprises about 0.1 to about 10% by weight Arnica extract. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Arnica extract.

In some embodiments, the composition comprises at least one extract derived from the group consisting of Harpagopthum, Bryophyllus pinnatum, Calendula officinalis, Boswellia serrata, Cinnamomum camphora, Mentha piperita, Ruta graveolens, and Symphytum officinale. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight of the least one extract derived from the group consisting of Harpagopthum, Bryophyllus pinnatum, Calendula officinalis, Boswellia serrata, Cinnamomum camphora, Mentha piperita, Ruta graveolens, and Symphytum officinale and combinations thereof.

In some embodiments, the composition comprises magnesium sulfate. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight magnesium sulfate.

In some embodiments, the composition comprises Capsicum oleoresin. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Capsicum oleoresin.

In some embodiments, the composition comprises cannabidiol (CBD). Cannabidiol is one of many active cannabinoids in Cannabis and constitutes up to about 40% of Cannabis sativa extracts. It is recognized as a major non-psychoactive cannabinoid, with a remarkable lack of any cognitive and psychoactive actions. Cannabidiol, 2-[3-methyl-6-(1-methylethenyl)-2-cyclohexen-1-yl]-5-pentyl-1,3-benzenediol, or (−)-trans-2-p-mentha-1,8-dien-3-yl-5-pentylresorcinol is generally shown with the following formula:

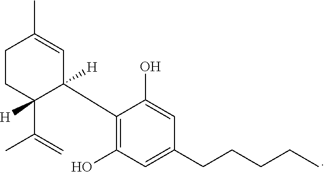 [SMILES: C=C(C)[C@@H]1CCC(C)=C[C@H]1c1c(O)cc(CCCCC)cc1O]

The cannabidiol may be a natural cannabidiol obtainable by extraction from a plant member of the genus Cannabis or any preparations of Cannabis (e.g., processed plant material). The natural cannabidiol may be extracted from any Cannabis sativa strain, such as hemp, or one of its preparations, e.g., marijuana, hashish, etc., according to methods known in the art.

The cannabidiol may also be a synthetic cannabidiol or a derivative or prodrug thereof which can be generated according to methods known in the art. Commercially available cannabidiol may be contaminated with delta 9-tetrahydrocannabinol. The presence of delta-9-tetrahydrocannabinol can be a concern because delta-9-tetrahydrocannabinol is regulated by the United States Drug Enforcement Administration as a Schedule I Drug. Therefore, substantially pure synthetically synthesized cannabidiol is preferred.

In some embodiments, the composition includes about 0.5 to about 2.0% by weight CBD. The composition may comprise about 0.5 to about 1.75% CBD, about 0.5 to about 1.5% CBD, about 0.5 to about 1.25% CBD, about 0.5 to about 1.0% CBD, about 0.5 to about 0.75% CBD, about 0.75 to about 2.0% CBD, about 0.75 to about 1.75% CBD, about 0.75 to about 1.5% CBD, about 0.75 to about 1.25% CBD, about 0.75 to about 1.0% CBD, about 1.0 to about 2.0% CBD, about 1.0 to about 1.25% CBD, about 1.0 to about 1.5% CBD, about 1.0 to about 1.75% CBD, about 1.25 to about 2.0% CBD, about 1.25 to about 1.75% CBD, about 1.25 to about 1.5% CBD, about 1.5 to about 2.0% CBD, about 1.5 to about 1.75% CBD, or about 1.75 to about 2.0% CBD. The composition may comprise about 0.5% CBD, about 0.6% CBD, about 0.7% CBD, about 0.8% CBD, about 0.9% CBD, about 1.0% CBD, about 1.1% CBD, about 1.2% CBD, about 1.3% CBD, about 1.4% CBD, about 1.5% CBD, about 1.6% CBD, about 1.7% CBD, about 1.8% CBD, about 1.9% CBD, or about 2.0% CBD.

### 3. Methods for Treating Foot Pain

The present disclosure provides methods for treating or preventing foot pain comprising administering to a subject a composition comprising 1% to 25% by weight pine resin.

a) Foot Pain

Both chronic and acute foot pain affect numerous people. Foot pain may be caused by many different diseases, deformities, biomechanical conditions, improper footwear, or injuries.

The foot pain may be caused by deformities, such as tarsal coalitions, calcaneal varus, calcaneal valgus, bunions, claw toes, mallet toes, hammertoes, and bone spurs. The foot pain may be caused by trauma from an acute injury or from accumulative repetitive injury, such as Achilles tendinitis or rupture. Injuries to the skin and internal structures may also be caused by small repetitive traumas or pressures. Micro-trauma injuries can be caused by running on uneven surfaces or surfaces that are too hard or too soft, or by wearing shoes that have poor force-absorption qualities, are not activity specific, or fit incorrectly. Pain in the forefoot may be caused by wearing shoes that are too tight or high heels. Pain may also be caused following periods of prolonged standing.

The foot pain may be caused by overstretching, overuse, or overloading of the muscles and fascia of the foot. For example, plantar fasciitis which commonly causes stabbing pain that usually occurs during the first steps of the morning or prolonged periods of inactivity.

The foot pain may be caused by injuries such as ligament sprains, muscle strains, bruises, and fractures. Sprains, strains, bruises, and fractures may be the result of a single or combination of stresses to the foot. In some embodiments the sprain may be an ankle sprain. Repeated overstressing of the same structure of the foot may cause stress fractures, tendonitis, plantar fasciitis, and acute and chronic osteoarthritis.

Other joint pain may also benefit from treatment with the composition described herein, including for example, knee, wrist or elbow pain.

The foot pain may also be caused by injury to the bones and joints of the foot from a single blow or twist to the foot or also by repetitive trauma that can result in a stress fracture. A blunt-force injury such as the foot being stepped on may result not only in a bruise (contusion) injury but also damage to the muscles and ligaments of the foot. Metatarsalgia is from the repetitive irritation of the joints of the ball of the foot. The term “stone bruise” is commonly referred to as a specific localized pain and tenderness of the ball of the foot. “Turf toe” is a common athletic injury in which the tendon and capsule under the joint at the base of the big toe is strained.

In some embodiments, the composition is administered before the onset of foot pain to prevent or delay the onset of foot pain. In some embodiments, the composition is administered after the onset of foot pain to relieve, improve or remove the foot pain. In some embodiments, the composition is administered before and after the onset of foot pain.

b) Compositions

The composition may include about 1 to about 25% by weight pine resin. The term “pine rosin,” “rosin,” and “gum rosin” may be synonymous with pine resin.

In some embodiments, the composition includes at least about 1% by weight pine resin, at least about 2% by weight pine resin, at least about 5% by weight pine resin, at least about 10% by weight pine resin, at least about 15% by weight pine resin, at least about 20% by weight pine resin, or at least about 25% by weight pine resin. In some embodiments, the composition includes less than about 25% by weight pine resin, less than about 20% by weight pine resin, less than about 15% by weight pine resin, less than about 10% by weight pine resin, less than about 5% by weight pine resin, or less than about 2% by weight pine resin. The composition may comprise about 1 to about 25% by weight pine resin, about 1 to about 20% by weight pine resin, about 1 to about 15% by weight pine resin, about 1 to about 10% by weight pine resin, about 1 to about 5% by weight pine resin, about 2 to about 25% by weight pine resin, about 2 to about 20% by weight pine resin, about 2 to about 15% by weight pine resin, about 2 to about 10% by weight pine resin, about 5 to about 25% by weight pine resin, about 5- to about 20% by weight pine resin, about 5 to about 15% by weight pine resin, about 5 to about 10% by weight pine resin, about 10 to about 25% by weight pine resin, about 10 to about 20% by weight pine resin, about 10 to about 15% by weight pine resin, about 15 to about 25% by weight pine resin, or about 15 to about 20% by weight pine resin. In certain embodiments, the composition comprises between about 5% and about 15% by weight pine resin.

Pine resin is a mixture of compounds, mainly composed of resin acids, lignans, terpenes and volatile fatty acid compounds. In some embodiments, the pine resin comprises at least about 30% abietic acid. The pine resin may comprise at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

In some embodiments, the pine resin comprises at least about 20% abietic acid. The pine resin may comprise at least about 21% abietic acid, at least about 22% abietic acid, at least about 23% abietic acid, at least about 24% abietic acid, at least about 25% abietic acid, at least about 26% abietic acid, at least about 27% abietic acid, at least about 28% abietic acid, at least about 29% abietic acid, at least about 30% abietic acid, at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 20 to about 60% abietic acid, about 20 to about 55% abietic acid, about 20 to about 50% abietic acid, about 20 to about 45% abietic acid, about 20 to about 40% abietic acid, about 20 to about 35% abietic acid, about 20 to about 30% abietic acid, about 20 to about 25% abietic acid, about 25 to about 60% abietic acid, about 25 to about 55% abietic acid, about 25 to about 50% abietic acid, about 25 to about 45% abietic acid, about 25 to about 40% abietic acid, about 25 to about 30% abietic acid, about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

Different species of pine may give rise to different mixtures of compounds in pine resin. In addition, the different species of pine grown in environmental conditions that impart different properties based on the particular growing region. The pine resin used in the composition may be derived from pine species from Central America, Caribbean islands (e.g., Cuba, the Bahamas, Turks and Caicos, Trinidad and Tobago, etc.) and Southeastern United States. As such, the pine resins used in the compositions of the present invention likely contain different components than those found in a pine or juniper plants found in Asia. In some embodiments, the pine resin is derived from Pinus caribaea (Caribbean pine), Pinus elliottii, or a combination thereof.

In some embodiments, the composition comprises about 0.1 to about 10% by weight Arnica extract. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Arnica extract.

In some embodiments, the composition comprises at least one extract derived from the group consisting of Harpagopthum, Bryophyllus pinnatum, Calendula officinalis, Boswellia serrata, Cinnamomum camphora, Mentha piperita, Ruta graveolens, and Symphytum officinale. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight of each of the least one extract derived from the group consisting of Harpagopthum, Bryophyllus pinnatum, Calendula officinalis, Boswellia serrata, Cinnamomum camphora, Mentha piperita, Ruta graveolens, and Symphytum officinale.

In some embodiments, the composition comprises magnesium sulfate. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight magnesium sulfate.

In some embodiments, the composition comprises Capsicum oleoresin. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Capsicum oleoresin.

In some embodiments, the composition comprises cannabidiol (CBD). Cannabidiol is one of many active cannabinoids in Cannabis and constitutes up to about 40% of Cannabis sativa extracts. It is recognized as a major non-psychoactive cannabinoid, with a remarkable lack of any cognitive and psychoactive actions. Cannabidiol, 2-[3-methyl-6-(1-methylethenyl)-2-cyclohexen-1-yl]-5-pentyl-1,3-benzenediol, or (−)-trans-2-p-mentha-1,8-dien-3-yl-5-pentylresorcinol is generally shown with the following formula:

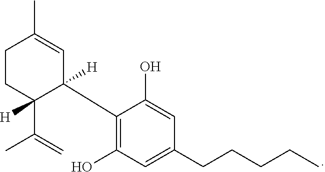 [SMILES: C=C(C)[C@@H]1CCC(C)=C[C@H]1c1c(O)cc(CCCCC)cc1O]

The cannabidiol may be a natural cannabidiol obtainable by extraction from a plant member of the genus Cannabis or any preparations of Cannabis (e.g., processed plant material). The natural cannabidiol may be extracted from any Cannabis sativa strain, such as hemp, or one of its preparations, e.g., marijuana, hashish, etc., according to methods known in the art.

The cannabidiol may also be a synthetic cannabidiol or a derivative or prodrug thereof which can be generated according to methods known in the art. Commercially available cannabidiol may be contaminated with delta 9-tetrahydrocannabinol. The presence of delta-9-tetrahydrocannabinol can be a concern because delta-9-tetrahydrocannabinol is regulated by the United States Drug Enforcement Administration as a Schedule I Drug. Therefore, substantially pure synthetically synthesized cannabidiol is preferred.

In some embodiments, the composition includes about 0.5 to about 2.0% by weight CBD. The composition may comprise about 0.5 to about 1.75% CBD, about 0.5 to about 1.5% CBD, about 0.5 to about 1.25% CBD, about 0.5 to about 1.0% CBD, about 0.5 to about 0.75% CBD, about 0.75 to about 2.0% CBD, about 0.75 to about 1.75% CBD, about 0.75 to about 1.5% CBD, about 0.75 to about 1.25% CBD, about 0.75 to about 1.0% CBD, about 1.0 to about 2.0% CBD, about 1.0 to about 1.25% CBD, about 1.0 to about 1.5% CBD, about 1.0 to about 1.75% CBD, about 1.25 to about 2.0% CBD, about 1.25 to about 1.75% CBD, about 1.25 to about 1.5% CBD, about 1.5 to about 2.0% CBD, about 1.5 to about 1.75% CBD, or about 1.75 to about 2.0% CBD. The composition may comprise about 0.5% CBD, about 0.6% CBD, about 0.7% CBD, about 0.8% CBD, about 0.9% CBD, about 1.0% CBD, about 1.1% CBD, about 1.2% CBD, about 1.3% CBD, about 1.4% CBD, about 1.5% CBD, about 1.6% CBD, about 1.7% CBD, about 1.8% CBD, about 1.9% CBD, or about 2.0% CBD.

### 4. Methods for Treating Inflammation and Itch

The present disclosure provides methods for treating or preventing inflammation or an inflammatory-related condition comprising administering to a subject a composition comprising 1% to 25% by weight pine resin. The present disclosure also provides methods for treating or preventing itch or pruritis comprising administering to a subject a composition comprising 1% to 25% by weight pine resin.

a) Inflammation and Inflammatory-Related Conditions

Inflammation involves the activation of the immune system in response to harmful stimuli, such as, e.g., a pathogen, infection, irritant, or damage to cells. As a stereotyped response, inflammation is a mechanism of innate immunity, as compared to adaptive immunity, which is specific for each pathogen. Inflammation can be classified as either acute or chronic. Generally speaking, acute inflammation is mediated by granulocytes, while chronic inflammation is mediated by mononuclear cells such as monocytes and lymphocytes.

The method of the present invention is useful for, but not limited to, the prevention and/or treatment of inflammation regardless of the underlying cause. The method of the present invention is also useful for, but not limited to, the prevention and/or treatment of inflammatory-related conditions or disorders.

In some embodiments, the inflammation comprises a tissue inflammation. Tissue inflammation is a chronic inflammation that is confined to a particular tissue. A tissue inflammation includes, but is not limited to, a skin inflammation, a muscle inflammation, a tendon inflammation, a ligament inflammation, a bone inflammation, or a cartilage inflammation.

In some embodiments, the inflammation may comprise or be caused by a skin or dermatological disorder, including but not limited to: acne, including acne vulgaris; a bullous phemigoid; a dermatitis or eczema, including for example atopic dermatitis, chronic actinic dermatitis, atopic eczema, contact eczema, xerotic eczema, seborrhoeic dermatitis, dyshidrotic eczema, discoid eczema, dermatitis herpetiformis, neurodermatitis, and statis dermatitis; hidradenitis suppurativa; lichen planus; psoriasis including plaque psoriasis, nail psoriasis, guttate psoriasis, scalp psoriasis, inverse psoriasis, pustular psoriasis, erythrodermis psoriasis, and psoriatic arthritis; rosacea; and scleroderma including morphea.

In some embodiments, the inflammation is caused by athletic injuries, arthritis and bursitis, foot pain or tired feet, inflammation (edema) in extremities resulting from diabetes or pregnancy, allergic reactions such as poison ivy and insect bites or stings, tendonitis, superficial cuts and scrapes, shaving (e.g., razor bumps), dry skin, burns (e.g., sunburns, first or second degree burns), urticaria, inflammation following surgery, or inflammation following aesthetic procedures (e.g., breast Augmentation, liposuction, body lifts, Botox, fillers microneedling, chemical peels, IPL photofacial, laser hair removal, and laser skin resurfacing).

b) Itch

The composition may further treat itch (also known as pruritis) in a subject. As used herein, the term “itch” or, more technically “pruritis” refers to the sensation that elicits a reflex response to scratch. Itch can be a symptom of a disease, disorder or infection, or itch can arise spontaneously, without an underlying or identifiable physiological cause, known as idiopathic pruritus. The itch may be transient or chronic.

In some embodiments, the itch may be related to an inflammatory disease or inflammation as described above, e.g., allergic reactions such as poison ivy and insect bites or stings, dry skin, burns (e.g., sunburns, first or second degree burns), psoriasis, urticaria, and a dermatitis or eczema. In some embodiments, the itch may be unrelated to inflammation or an inflammatory disease, e.g., fungal infections including athlete's foot, yeast infections including diaper rash, parasitic infestations including scabies and lice, itch secondary to medications, senile itch or pruritus, wound healing, and chicken pox. The methods described are also applicable to itch with no known etiology.

c) Compositions

The composition may include about 1 to about 25% by weight pine resin. The term “pine rosin,” “rosin,” and “gum rosin” may be synonymous with pine resin.

In some embodiments, the composition includes at least about 1% by weight pine resin, at least about 2% by weight pine resin, at least about 5% by weight pine resin, at least about 10% by weight pine resin, at least about 15% by weight pine resin, at least about 20% by weight pine resin, or at least about 25% by weight pine resin. In some embodiments, the composition includes less than about 25% by weight pine resin, less than about 20% by weight pine resin, less than about 15% by weight pine resin, less than about 10% by weight pine resin, less than about 5% by weight pine resin, or less than about 2% by weight pine resin. The composition may comprise about 1 to about 25% by weight pine resin, about 1 to about 20% by weight pine resin, about 1 to about 15% by weight pine resin, about 1 to about 10% by weight pine resin, about 1 to about 5% by weight pine resin, about 2 to about 25% by weight pine resin, about 2 to about 20% by weight pine resin, about 2 to about 15% by weight pine resin, about 2 to about 10% by weight pine resin, about 5 to about 25% by weight pine resin, about 5- to about 20% by weight pine resin, about 5 to about 15% by weight pine resin, about 5 to about 10% by weight pine resin, about 10 to about 25% by weight pine resin, about 10 to about 20% by weight pine resin, about 10 to about 15% by weight pine resin, about 15 to about 25% by weight pine resin, or about 15 to about 20% by weight pine resin. In certain embodiments, the composition comprises between about 5% and about 15% by weight pine resin.

Pine resin is a mixture of compounds, mainly composed of resin acids, lignans, terpenes and volatile fatty acid compounds. In some embodiments, the pine resin comprises at least about 30% abietic acid. The pine resin may comprise at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

In some embodiments, the pine resin comprises at least about 20% abietic acid. The pine resin may comprise at least about 21% abietic acid, at least about 22% abietic acid, at least about 23% abietic acid, at least about 24% abietic acid, at least about 25% abietic acid, at least about 26% abietic acid, at least about 27% abietic acid, at least about 28% abietic acid, at least about 29% abietic acid, at least about 30% abietic acid, at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 20 to about 60% abietic acid, about 20 to about 55% abietic acid, about 20 to about 50% abietic acid, about 20 to about 45% abietic acid, about 20 to about 40% abietic acid, about 20 to about 35% abietic acid, about 20 to about 30% abietic acid, about 20 to about 25% abietic acid, about 25 to about 60% abietic acid, about 25 to about 55% abietic acid, about 25 to about 50% abietic acid, about 25 to about 45% abietic acid, about 25 to about 40% abietic acid, about 25 to about 30% abietic acid, about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

Different species of pine may give rise to different mixtures of compounds in pine resin. In addition, the different species of pine grown in environmental conditions that impart different properties based on the particular growing region. The pine resin used in the composition may be derived from pine species from Central America, Caribbean islands (e.g., Cuba, the Bahamas, Turks and Caicos, Trinidad and Tobago, etc.) and Southeastern United States. As such, the pine resins used in the compositions of the present invention likely contain different components than those found in a pine or juniper plants found in Asia. In some embodiments, the pine resin is derived from Pinus caribaea (Caribbean pine), Pinus elliottii, or a combination thereof.

In some embodiments, the composition comprises about 0.1 to about 10% by weight Arnica extract. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Arnica extract.

In some embodiments, the composition comprises at least one extract derived from the group consisting of Harpagopthum, Bryophyllus pinnatum, Calendula officinalis, Boswellia serrata, Cinnamomum camphora, Mentha piperita, Ruta graveolens, and Symphytum officinale. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight of each of the least one extract derived from the group consisting of Harpagopthum, Bryophyllus pinnatum, Calendula officinalis, Boswellia serrata, Cinnamomum camphora, Mentha piperita, Ruta graveolens, and Symphytum officinale and combinations thereof.

In some embodiments, the composition comprises magnesium sulfate. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight magnesium sulfate.

In some embodiments, the composition comprises Capsicum oleoresin. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Capsicum oleoresin.

In some embodiments, the composition comprises cannabidiol (CBD). Cannabidiol is one of many active cannabinoids in Cannabis and constitutes up to about 40% of Cannabis sativa extracts. It is recognized as a major non-psychoactive cannabinoid, with a remarkable lack of any cognitive and psychoactive actions. Cannabidiol, 2-[3-methyl-6-(1-methylethenyl)-2-cyclohexen-1-yl]-5-pentyl-1,3-benzenediol, or (−)-trans-2-p-mentha-1,8-dien-3-yl-5-pentylresorcinol is generally shown with the following formula:

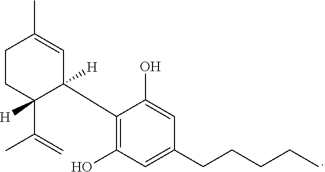 [SMILES: C=C(C)[C@@H]1CCC(C)=C[C@H]1c1c(O)cc(CCCCC)cc1O]

The cannabidiol may be a natural cannabidiol obtainable by extraction from a plant member of the genus Cannabis or any preparations of Cannabis (e.g., processed plant material). The natural cannabidiol may be extracted from any Cannabis sativa strain, such as hemp, or one of its preparations, e.g., marijuana, hashish, etc., according to methods known in the art.

The cannabidiol may also be a synthetic cannabidiol or a derivative or prodrug thereof which can be generated according to methods known in the art. Commercially available cannabidiol may be contaminated with delta 9-tetrahydrocannabinol. The presence of delta-9-tetrahydrocannabinol can be a concern because delta-9-tetrahydrocannabinol is regulated by the United States Drug Enforcement Administration as a Schedule I Drug. Therefore, substantially pure synthetically synthesized cannabidiol is preferred.

In some embodiments, the composition includes about 0.5 to about 2.0% by weight CBD. The composition may comprise about 0.5 to about 1.75% CBD, about 0.5 to about 1.5% CBD, about 0.5 to about 1.25% CBD, about 0.5 to about 1.0% CBD, about 0.5 to about 0.75% CBD, about 0.75 to about 2.0% CBD, about 0.75 to about 1.75% CBD, about 0.75 to about 1.5% CBD, about 0.75 to about 1.25% CBD, about 0.75 to about 1.0% CBD, about 1.0 to about 2.0% CBD, about 1.0 to about 1.25% CBD, about 1.0 to about 1.5% CBD, about 1.0 to about 1.75% CBD, about 1.25 to about 2.0% CBD, about 1.25 to about 1.75% CBD, about 1.25 to about 1.5% CBD, about 1.5 to about 2.0% CBD, about 1.5 to about 1.75% CBD, or about 1.75 to about 2.0% CBD. The composition may comprise about 0.5% CBD, about 0.6% CBD, about 0.7% CBD, about 0.8% CBD, about 0.9% CBD, about 1.0% CBD, about 1.1% CBD, about 1.2% CBD, about 1.3% CBD, about 1.4% CBD, about 1.5% CBD, about 1.6% CBD, about 1.7% CBD, about 1.8% CBD, about 1.9% CBD, or about 2.0% CBD.

### 5. Methods for Repelling an Insect

The present disclosure provides methods for repelling an insect comprising applying to a subject, in an amount effective to repel the insect, a composition comprising 1% to 25% by weight pine resin.

a) Repelling an Insect

Traditional insect control methods often involve the heavy use of chemical insecticides that are harmful to the environment and often to human health. Moreover, insects can develop resistance to these chemicals, suggesting that there is a need to identify novel ways of insect control that are effective and environmentally friendly.

As used herein, the term “insect” refers to any member of a large group of invertebrate animals characterized, in the adult state (non-adult insect states include larva and pupa) by division of the body into head, thorax, and abdomen, three pairs of legs, and, often (but not always) two pairs of membranous wings. This definition therefore includes a variety of biting insects (e.g., ants, bees, black flies, chiggers, fleas, green head flies, mosquitoes, stable flies, ticks, wasps), wood-boring insects (e.g., termites), noxious insects (e.g., houseflies, cockoaches, lice, roaches, wood lice), and household pests (e.g., flour and bean beetles, dust mites, moths, silverfish, weevils).

In some embodiments, the composition is applied to a subject's skin. in some embodiments, the composition is applied to an article worn or inhabited by the subject. The article may include: garments, belts, collars, or other articles worn or used by the subject from whom insects are to be repelled, bedding, netting, screens, camping gear and the like. It will be appreciated that the application of the compositions and compounds of the disclosure do not only include human subjects but include any animal subject to biting or affected by insects (e.g., canines, equines, bovines and other animals).

b) Compositions

The composition may include about 1 to about 25% by weight pine resin. The term “pine rosin,” “rosin,” and “gum rosin” may be synonymous with pine resin.

In some embodiments, the composition includes at least about 1% by weight pine resin, at least about 2% by weight pine resin, at least about 5% by weight pine resin, at least about 10% by weight pine resin, at least about 15% by weight pine resin, at least about 20% by weight pine resin, or at least about 25% by weight pine resin. In some embodiments, the composition includes less than about 25% by weight pine resin, less than about 20% by weight pine resin, less than about 15% by weight pine resin, less than about 10% by weight pine resin, less than about 5% by weight pine resin, or less than about 2% by weight pine resin. The composition may comprise about 1 to about 25% by weight pine resin, about 1 to about 20% by weight pine resin, about 1 to about 15% by weight pine resin, about 1 to about 10% by weight pine resin, about 1 to about 5% by weight pine resin, about 2 to about 25% by weight pine resin, about 2 to about 20% by weight pine resin, about 2 to about 15% by weight pine resin, about 2 to about 10% by weight pine resin, about 5 to about 25% by weight pine resin, about 5- to about 20% by weight pine resin, about 5 to about 15% by weight pine resin, about 5 to about 10% by weight pine resin, about 10 to about 25% by weight pine resin, about 10 to about 20% by weight pine resin, about 10 to about 15% by weight pine resin, about 15 to about 25% by weight pine resin, or about 15 to about 20% by weight pine resin. In certain embodiments, the composition comprises between about 5% and about 15% by weight pine resin.

Pine resin is a mixture of compounds, mainly composed of resin acids, lignans, terpenes and volatile fatty acid compounds. In some embodiments, the pine resin comprises at least 30% abietic acid. In some embodiments, the pine resin comprises at least about 30% abietic acid. The pine resin may comprise at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

In some embodiments, the pine resin comprises at least about 20% abietic acid. The pine resin may comprise at least about 21% abietic acid, at least about 22% abietic acid, at least about 23% abietic acid, at least about 24% abietic acid, at least about 25% abietic acid, at least about 26% abietic acid, at least about 27% abietic acid, at least about 28% abietic acid, at least about 29% abietic acid, at least about 30% abietic acid, at least about 31% abietic acid, at least about 32% abietic acid, at least about 33% abietic acid, at least about 34% abietic acid, at least about 35% abietic acid, at least about 36% abietic acid, at least about 37% abietic acid, at least about 38% abietic acid, at least about 39% abietic acid, at least about 40% abietic acid, at least about 41% abietic acid, at least about 42% abietic acid, at least about 43% abietic acid, at least about 44% abietic acid, at least about 45% abietic acid, at least about 46% abietic acid, at least about 47% abietic acid, at least about 48% abietic acid, at least about 49% abietic acid, or at least about 50% abietic acid. The pine resin may comprise about 20 to about 60% abietic acid, about 20 to about 55% abietic acid, about 20 to about 50% abietic acid, about 20 to about 45% abietic acid, about 20 to about 40% abietic acid, about 20 to about 35% abietic acid, about 20 to about 30% abietic acid, about 20 to about 25% abietic acid, about 25 to about 60% abietic acid, about 25 to about 55% abietic acid, about 25 to about 50% abietic acid, about 25 to about 45% abietic acid, about 25 to about 40% abietic acid, about 25 to about 30% abietic acid, about 30 to about 60% abietic acid, about 30 to about 55% abietic acid, about 30 to about 50% abietic acid, about 30 to about 45% abietic acid, about 30 to about 40% abietic acid, about 30 to about 35% abietic acid, about 35 to about 60% abietic acid, about 35 to about 55% abietic acid, about 35 to about 50% abietic acid, about 35 to about 45% abietic acid, about 35 to about 40% abietic acid, about 40 to about 60% abietic acid, about 40 to about 55% abietic acid, about 40 to about 50% abietic acid, about 40 to about 45% abietic acid, about 45 to about 60% abietic acid, about 45 to about 55% abietic acid, about 45 to about 50% abietic acid, about 50 to about 60% abietic acid, about 50 to about 55% abietic acid, or about 55 to about 60% abietic acid.

Different species of pine may give rise to different mixtures of compounds in pine resin. In addition, the different species of pine grown in environmental conditions that impart different properties based on the particular growing region. The pine resin used in the composition may be derived from pine species from Central America, Caribbean islands (e.g., Cuba, the Bahamas, Turks and Caicos, Trinidad and Tobago, etc.) and Southeastern United States. As such, the pine resins used in the compositions of the present invention likely contain different components than those found in a pine or juniper plants found in Asia. In some embodiments, the pine resin is derived from Pinus caribaea (Caribbean pine), Pinus elliottii, or a combination thereof.

In some embodiments, the composition comprises about 0.1 to about 10% by weight Arnica extract. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Arnica extract.

In some embodiments, the composition comprises at least one extract derived from the group consisting of Harpagopthum, Bryophyllus pinnatum, Calendula officinalis, Boswellia serrata, Cinnamomum camphora, Mentha piperita, Ruta graveolens, and Symphytum officinale and combinations thereof. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight of each of the least one extract derived from the group consisting of Harpagopthum, Bryophyllus pinnatum, Calendula officinalis, Boswellia serrata, Cinnamomum camphora, Mentha piperita, Ruta graveolens, and Symphytum officinale and combinations thereof.

In some embodiments, the composition comprises magnesium sulfate. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight magnesium sulfate.

In some embodiments, the composition comprises Capsicum oleoresin. The composition may include between about 0.1 to about 10%, between about 0.1 to about 9%, between about 0.1 to about 8%, between about 0.1 to about 7%, between about 0.1 to about 6%, between about 0.1 to about 5%, between about 0.1 to about 4%, between about 0.1 to about 3%, between about 0.1 to about 2%, between about 0.1 to about 1%, between about 0.5 to about 10%, between about 0.5 to about 9%, between about 0.5 to about 8%, between about 0.5 to about 7%, between about 0.5 to about 6%, between about 0.5 to about 5%, between about 0.5 to about 4%, between about 0.5 to about 3%, between about 0.5 to about 2%, between about 0.5 to about 1%, between about 1 to about 10%, between about 1 to about 9%, between about 1 to about 8%, between about 1 to about 7%, between about 1 to about 6%, between about 1 to about 5%, between about 1 to about 4%, between about 1 to about 3%, between about 1 to about 2%, between about 2 to about 10%, between about 2 to about 9%, between about 2 to about 8%, between about 2 to about 7%, between about 2 to about 6%, between about 2 to about 5%, between about 2 to about 4%, between about 2 to about 3%, between about 3 to about 10%, between about 3 to about 9%, between about 3 to about 8%, between about 3 to about 7%, between about 3 to about 6%, between about 3 to about 5%, between about 3 to about 4%, between about 4 to about 10%, between about 4 to about 9%, between about 4 to about 8%, between about 4 to about 7%, between about 4 to about 6%, between about 4 to about 5%, between about 5 to about 10%, between about 5 to about 9%, between about 5 to about 8%, between about 5 to about 7%, between about 5 to about 6%, between about 6 to about 10%, between about 6 to about 9%, between about 6 to about 8%, between about 6 to about 7%, between about 7 to about 10%, between about 7 to about 9%, between about 7 to about 8%, between about 8 to about 10%, between about 8 to about 9%, or between about 9 to about 10% by weight Capsicum oleoresin.

In some embodiments, the composition comprises cannabidiol (CBD). Cannabidiol is one of many active cannabinoids in Cannabis and constitutes up to about 40% of Cannabis sativa extracts. It is recognized as a major non-psychoactive cannabinoid, with a remarkable lack of any cognitive and psychoactive actions. Cannabidiol, 2-[3-methyl-6-(1-methylethenyl)-2-cyclohexen-1-yl]-5-pentyl-1,3-benzenediol, or (−)-trans-2-p-mentha-1,8-dien-3-yl-5-pentylresorcinol is generally shown with the following formula:

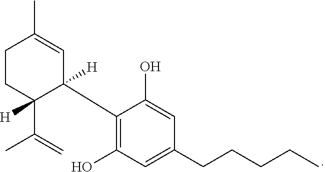 [SMILES: C=C(C)[C@@H]1CCC(C)=C[C@H]1c1c(O)cc(CCCCC)cc1O]

The cannabidiol may be a natural cannabidiol obtainable by extraction from a plant member of the genus Cannabis or any preparations of Cannabis (e.g., processed plant material). The natural cannabidiol may be extracted from any Cannabis sativa strain, such as hemp, or one of its preparations, e.g., marijuana, hashish, etc., according to methods known in the art.

The cannabidiol may also be a synthetic cannabidiol or a derivative or prodrug thereof which can be generated according to methods known in the art. Commercially available cannabidiol may be contaminated with delta 9-tetrahydrocannabinol. The presence of delta-9-tetrahydrocannabinol can be a concern because delta-9-tetrahydrocannabinol is regulated by the United States Drug Enforcement Administration as a Schedule I Drug. Therefore, substantially pure synthetically synthesized cannabidiol is preferred.

In some embodiments, the composition includes about 0.5 to about 2.0% by weight CBD. The composition may comprise about 0.5 to about 1.75% CBD, about 0.5 to about 1.5% CBD, about 0.5 to about 1.25% CBD, about 0.5 to about 1.0% CBD, about 0.5 to about 0.75% CBD, about 0.75 to about 2.0% CBD, about 0.75 to about 1.75% CBD, about 0.75 to about 1.5% CBD, about 0.75 to about 1.25% CBD, about 0.75 to about 1.0% CBD, about 1.0 to about 2.0% CBD, about 1.0 to about 1.25% CBD, about 1.0 to about 1.5% CBD, about 1.0 to about 1.75% CBD, about 1.25 to about 2.0% CBD, about 1.25 to about 1.75% CBD, about 1.25 to about 1.5% CBD, about 1.5 to about 2.0% CBD, about 1.5 to about 1.75% CBD, or about 1.75 to about 2.0% CBD. The composition may comprise about 0.5% CBD, about 0.6% CBD, about 0.7% CBD, about 0.8% CBD, about 0.9% CBD, about 1.0% CBD, about 1.1% CBD, about 1.2% CBD, about 1.3% CBD, about 1.4% CBD, about 1.5% CBD, about 1.6% CBD, about 1.7% CBD, about 1.8% CBD, about 1.9% CBD, or about 2.0% CBD.

### 6. Administration

The pine resin compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the composition may be formulated for the desired administration method. Techniques and formulations may generally be found in “Remington's Pharmaceutical Sciences,” (Meade Publishing Co., Easton, Pa.). The route by which the disclosed compounds are administered, and the form of the composition will dictate the type of carrier to be used. The composition may be in a variety of forms, suitable, for example, for topical administration (e.g., dermal, pulmonary, nasal, aural, ocular, liposome delivery systems, or iontophoresis) or oral administration.

Compositions for oral administration can have various dosage forms. Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include the active ingredients and a carrier, namely, a carrier selected from diluents, colorants, flavors, sweeteners, preservatives, solvents, suspending agents, and surfactants.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0 to about 100%.

The disclosed compositions can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. The carrier of the topical composition preferably aids penetration of the compounds into the skin. The carrier may further include one or more optional components.

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.1%, particularly, about 0.001% to about 0.5%. The fragrances may include essential oils, including, for example, wintergreen oil, eucalyptus oil, and juniper oil 101301 Suitable pH adjusting additives include HCl or NaOH in amounts sufficient to adjust the pH of a topical pharmaceutical composition.

In some embodiments, the composition is an ointment, cream, gel, lotion, spray, foam, paste, suspension or dispersion for topical administration.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A method for treating or preventing a disease or disorder, the method comprising administering to a subject a composition comprising about 1% to about 25% by weight pine resin,
wherein the disease or disorder is selected from the group consisting of: muscle soreness, foot pain, inflammation or an inflammatory-related condition, and itch.

2. The method of claim 1, wherein the composition comprises between about 2% and about 20% by weight pine resin.

3. The method of claim 1, wherein the pine resin comprises at least about 20% abietic acid.

4. The method of claim 1, wherein the pine resin comprises at least about 30% abietic acid.

5. The method of claim 1, wherein the pine resin is derived from Pinus caribaea, Pinus elliottii, or a combination thereof.

6. The method of claim 1, wherein the composition further comprises about 0.1 about 10% by weight Arnica extract.

7. The method of claim 1, wherein the composition further comprises at least one extract derived from the group consisting of Harpagopthum, Bryophyllus pinnatum, Calendula officinalis, Boswellia serrata, Cinnamomum camphora, Mentha piperita, Ruta graveolens, magnesium sulfate, Capsicum oleoresin, and Symphytum officinale and combinations thereof.

8. The method of claim 1, wherein the composition further comprises magnesium sulfate.

9. The method of claim 1, wherein the composition further comprises Capsicum oleoresin.

10. The method of claim 1, wherein the composition further comprises cannabidiol (CBD).

11. The method of claim 1, wherein the composition is an ointment, cream, gel, lotion, spray, foam, paste, suspension or dispersion for topical administration.

12. The method of claim 1, wherein the composition is administered before onset of the disease or disorder.

13. The method of claim 1, wherein the composition is administered after the onset of the disease or disorder.

14. The method of claim 1, wherein the muscle soreness is delayed onset muscle soreness, acute exercise-induced muscle soreness, a muscle cramp, or a sprain.

15. The method of claim 1, wherein the foot pain comprises deformities, overuse, injuries, or a combination thereof.

16. The method of claim 1, wherein the inflammation or inflammatory-related condition comprises tissue inflammation.

17. The method of claim 1, wherein the inflammation or inflammatory-related condition comprises a skin disorder, inflammation caused by athletic injuries, arthritis, bursitis, foot pain, inflammation in extremities resulting from diabetes or pregnancy, allergic reactions, tendonitis, superficial cuts and scrapes, shaving, dry skin, bums, inflammation following surgery, or inflammation following aesthetic procedures.

18. A method of repelling an insect, comprising applying to a subject, in an amount effective to repel the insect, a composition comprising about 1% to about 25% by weight pine resin.

19. The method of claim 18, wherein the composition comprises between about 2% and about 20% by weight pine resin.

20. The method of claim 18, wherein the pine resin comprises at least about 20% abietic acid.

21. The method of claim 18, wherein the pine resin comprises at least about 30% abietic acid.

22. The method of claim 18, wherein the pine resin is derived from Pinus caribaea, Pinus elliottii, or a combination thereof.

23. The method of claim 18, wherein the composition further comprises about 0.1 to about 10% by weight Arnica extract.

24. The method of claim 18, wherein the composition further comprises at least one extract derived from the group consisting of Harpagopthum, Bryophyllus pinnatum, Calendula officinalis, Boswellia serrata, Cinnamomum camphora, Mentha piperita, Ruta graveolens, magnesium sulfate, Capsicum oleoresin, and Symphytum officinale and combinations thereof.

25. The method of claim 18, wherein the composition further comprises magnesium sulfate.

26. The method of claim 18, wherein the composition further comprises Capsicum oleoresin.

27. The method of claim 18, wherein the composition further comprises cannabidiol (CBD).

28. The method of claim 18, wherein the composition is an ointment, cream, gel, lotion, spray, foam, paste, suspension or dispersion for topical administration.

29. The method of claim 18, wherein the applying comprises applying the composition to the skin of the subject.

30. The method of claim 18, wherein the applying comprises applying the composition to an article, wherein the article is worn or inhabited by the subject.
